(19) United States
(12) Patent Application Publication
(10) Pub. No.: US 2026/0328635 A1
(43) Pub. Date: Oct. 22, 2026

(54) DEVICE AND METHOD FOR CALIBRATING A FORCE PLATE

(71) Applicant: Ohio State Innovation Foundation, Columbus, OH (US)

(72) Inventors: James ONATE, Powell, OH (US); Yun Seok KANG, Dublin, OH (US); Jaclyn CACCESE, Powell, OH (US); Joshua HAGEN, Cincinnati, OH (US); Nathan EDWARDS, Columbus, OH (US)

(21) Appl. No.: 19/105,574

(22) PCT Filed: Aug. 23, 2023
(86) PCT No.: PCT/US2023/030924

§ 371 Date: Feb. 21, 2025

Related U.S. Application Data
(60) Provisional application No. 63/400,214, filed on Aug. 23, 2022.

(51) Int. Cl. G01L 25/00

(52) U.S. Cl. CPC G01L 25/00

(57) ABSTRACT

An example device for calibrating a force plate includes a frame including a base configured to contact a surface on which the force plate is supported and one or more supports extending from the base; a calibration ball; and a releaser coupled to the one or more supports, the releaser further being couplable to the calibration ball, where the releaser is actuatable from a first condition in which the calibration ball is coupled to the one or more supports and a second condition in which the calibration ball is uncoupled from the one or more supports. Example methods of calibrating a force plate include calibrating the force plate using one or more test results obtained by measuring the force of the calibration ball in different test conditions.

### CROSS-REFERENCE TO RELATED APPLICATIONS

This application claims the benefit of U.S. provisional patent application No. 63/400,214 filed on Aug. 23, 2022, and titled “DEVICE AND METHOD FOR CALIBRATING A FORCE PLATE,” the disclosure of which is expressly incorporated herein by reference in its entirety.

### BACKGROUND

Force measurement can be used to understand and characterize physical processes. When studying real-world processes, force measurement can be difficult because forces may act in different directions and over different distances and areas. Designers of force measurement systems can use force plates to provide a surface for a force to act on. However, force plates can move when they are not secured to the surface, which can reduce the accuracy of the force plates. To overcome this problem, force plates can be attached to surfaces, or embedded in surfaces, so that the plate is fixed relative to those surfaces. Fixing the plate in place can increase the accuracy of the force measurement, but prevents the plate from being easily moved to different real-world locations to perform measurements.

### SUMMARY

In some aspects, the techniques described herein relate to a device for calibrating a force plate, the device including: a frame including a base configured to contact a surface on which the force plate is supported and one or more supports extending from the base; a calibration ball; and a releaser coupled to the one or more supports, the releaser further being couplable to the calibration ball, wherein the releaser is actuatable from a first condition in which the calibration ball is coupled to the one or more supports and a second condition in which the calibration ball is uncoupled from the one or more supports.

In some aspects, the techniques described herein relate to a device, wherein the releaser is a latch.

In some aspects, the techniques described herein relate to a device, wherein the latch is remotely actuatable.

In some aspects, the techniques described herein relate to a device, wherein actuation of the latch from the first condition to the second condition is performable at a repeatable speed.

In some aspects, the techniques described herein relate to a device, wherein the releaser is a magnet.

In some aspects, the techniques described herein relate to a device, further including a cable coupled to the calibration ball.

In some aspects, the techniques described herein relate to a device, wherein a distance from the base to the releaser is adjustable.

In some aspects, the techniques described herein relate to a device, wherein the calibration ball is 20 lb to 30 lb.

In some aspects, the techniques described herein relate to a method of calibrating a force plate, the method including: obtaining a device for calibrating a force plate, the device including: a frame including a base configured to contact a surface on which the force plate is supported and one or more supports extending from the base; a calibration ball; and a releaser coupled to the one or more supports, the releaser further being couplable to the calibration ball, wherein the releaser is actuatable from a first condition in which the calibration ball is coupled to the one or more supports and a second condition in which the calibration ball is uncoupled from the one or more supports; disposing the base of the frame of the device over a force plate such that the force plate is directly below the calibration ball coupled to the releaser in the first condition; actuating the releaser from the first condition to the second condition such that the calibration ball is caused to fall from the releaser onto the force plate; and adjusting a calibration setting of the force plate to calibrate the force plate.

In some aspects, the techniques described herein relate to a method, further including, after the calibration ball is caused to fall from the releaser onto the force plate, comparing a force measurement from the force plate to an expected force measurement based on the device.

In some aspects, the techniques described herein relate to a method, further including, before disposing the base of the frame of the device, calibrating the device to establish an expected force measurement.

In some aspects, the techniques described herein relate to a method, wherein the releaser is a latch.

In some aspects, the techniques described herein relate to a method, wherein the latch is remotely actuatable.

In some aspects, the techniques described herein relate to a method, wherein actuation of the latch from the first condition to the second condition is performable at a repeatable speed.

In some aspects, the techniques described herein relate to a method, wherein the releaser is a magnet.

In some aspects, the techniques described herein relate to a method, wherein the device further includes a cable coupled to the calibration ball.

In some aspects, the techniques described herein relate to a method, wherein a distance from the base to the releaser is adjustable.

In some aspects, the techniques described herein relate to a method, further including, before adjusting a calibration setting, adjusting the distance from the base to the releaser, and actuating the releaser from the first condition to the second condition such that the calibration ball is caused to fall from the releaser onto the force plate at the adjusted distance.

In some aspects, the techniques described herein relate to a method, wherein the calibration ball is 20 lb to 30 lb.

In some aspects, the techniques described herein relate to a method, wherein the calibration ball is a first calibration ball and the device further includes a second calibration ball having a different weight than the first calibration ball, wherein the method further includes, before adjusting a calibration setting, actuating the releaser from the first condition to the second condition such that the second calibration ball is caused to fall from the releaser onto the force plate.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

FIG. 1 illustrates a device for calibrating a force plate, according to implementations of the present disclosure.

FIG. 2 illustrates a device including a static force plate, according to implementations of the present disclosure.

FIG. 3 illustrates a block diagram of a force plate, according to implementations of the present disclosure.

FIG. 4 illustrates a method for calibrating a force plate, according to implementations of the present disclosure.

FIG. 5 illustrates a perspective view of a device for calibrating a force plate, according to implementations of the present disclosure.

FIG. 6A illustrates a top view of the device illustrated in FIG. 5.

FIG. 6B illustrates a first side view of the device illustrated in FIG. 5.

FIG. 6C illustrates a second side view of the device illustrated in FIG. 5.

FIG. 6D illustrates a perspective view of the device shown in FIGS. 5-6C.

FIG. 7 illustrates an example computing device.

FIG. 8 illustrates an example 15 lb medicine ball and 11 lb medicine ball that were used in a study of an example implementation of the present disclosure.

FIG. 9A illustrates example locations where a weight can be dropped on a force plate, according to implementations of the present disclosure.

FIG. 9B illustrates example force measurements when weights are dropped in the locations shown in FIG. 9A.

FIG. 10A illustrates results of force tests for different force plate locations where drop tests were conducted using a 15 lb medicine ball, according to implementations of the present disclosure.

FIG. 10B illustrates results of force tests for different force plate locations where drop tests were performed using an 11 lb medicine ball, according to implementations of the present disclosure.

FIG. 11 illustrates force-time curves for an 11 lb weight without scaling, according to implementations of the present disclosure.

FIG. 12A illustrates force-time curves for an 11 lb weight with a scaled Y-axis.

FIG. 12B illustrates Y-axis scaling factors that were applied to the curves shown in FIG. 12A.

FIG. 13A illustrates force-time curves for an 11 lb weight with a scaled X-axis, according to implementations of the present disclosure.

FIG. 13B illustrates the X-axis scaling factors that were applied to the curves shown in FIG. 13A.

FIG. 14 illustrates force-time curves for a 15 lb weight without scaling, according to implementations of the present disclosure.

FIG. 15A illustrates force time-curves for the 15 lb weight with a scaled Y-axis, according to implementations of the present disclosure.

FIG. 15B illustrates the Y-axis scaling factors that were applied to the curves shown in FIG. 15A.

FIG. 16A illustrates force-time curves for the 15 lb weight with a scaled X-axis and a scaled Y-axis, according to implementations of the present disclosure.

FIG. 16B illustrates the X-axis scaling factors that were applied to the curves shown in FIG. 16A.

FIG. 17A illustrates force-time curves for an 11 lb weight aligned using the first instance of 50 N on the respective curves, according to implementations of the present disclosure.

FIG. 17B illustrates force time curves for a 15 lb weight aligned using the first instance of 50 N on the respective curves, according to implementations of the present disclosure.

FIG. 18A illustrates force time curves for an 11 lb weight aligned using the first instance of 200 N on the respective curves, according to implementations of the present disclosure.

FIG. 18B illustrates force time curves for a 15 lb weight aligned using the first instance of 200 N on the respective curves, according to implementations of the present disclosure.

FIG. 19A illustrates force-time curves for an 11 lb weight, where the curves aligned using 40% of the maximum force for the embedded force plate, according to implementations of the present disclosure.

FIG. 19B illustrates force-time curves for an 15 lb weight, where the curves aligned using 40% of the maximum force for the embedded force plate, according to implementations of the present disclosure.

FIG. 20 illustrates a table of intraclass correlation coefficients and root-mean-square error values for example implementations of the present disclosure.

### DETAILED DESCRIPTION

Implementations of the present disclosure include force plates that can be used on different surfaces, including non-rigid surfaces. Existing force plates are often designed to be used on a rigid surface, but users often apply them to a variety of surfaces in practice (e.g. grass). Current force plate validation methods do not address calibration on a variety of surfaces.

The devices, systems, and methods disclosed herein provide for calibration of any force plate from any manufacturer to be applied (i.e. adjusting forces and moments) on any surface. A device for calibrating a force plate includes a calibration ball suspended from a predetermined distance. The calibration ball can be dropped onto a first force plate that is known to be calibrated (e.g., a force plate that is disposed on a stable and static surface). The resulting force reading for the calibration ball dropped onto the known first force plate can act as an expected reading.

The device can then be transferred to a second force plate at any location, such as one on grass or on soil. The device can be positioned above the second force plate and the calibration ball can again be dropped from the same height. The reading from the calibration ball being dropped onto the second force plate can be compared to the expected reading. The difference between the second reading and the expected reading can be used to calibrate the second force plate to the surface on which it is disposed.

In some instances, multiple expected readings can be made using different weight calibration balls and/or by dropping the calibration ball(s) from different heights. The drops of multiple weight calibration balls and/or different heights can then be performed on the second force plate and calibration of the second force plate can be done based on the differences in force readings from each of the drops.

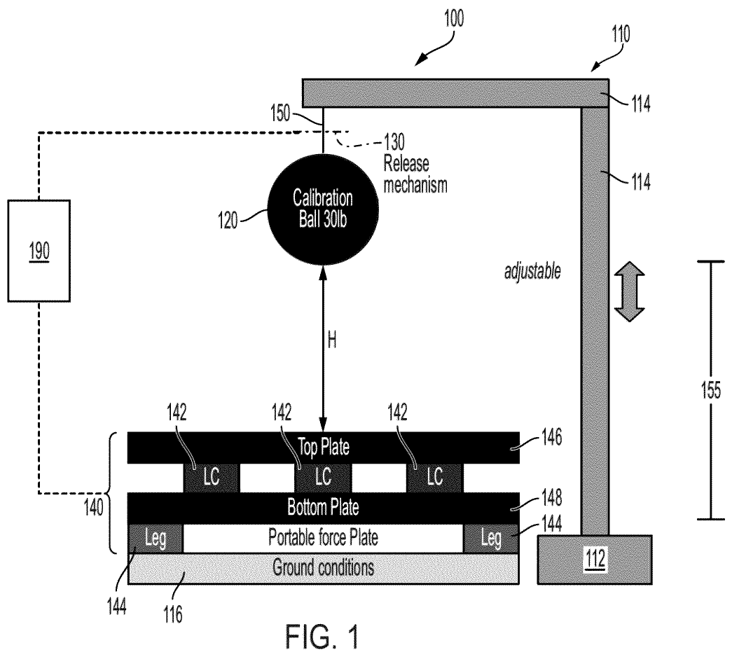

With reference to FIG. 1, an example device 100 for calibrating a force plate is shown according to an implementation of the present disclosure.

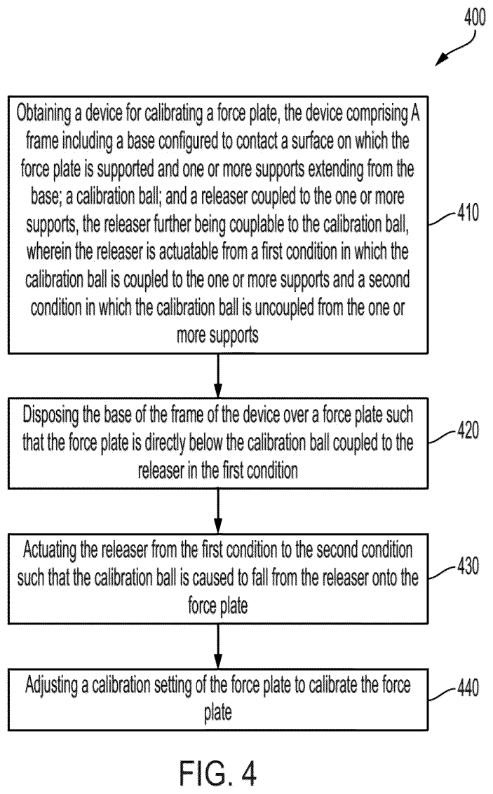

The device 100 includes a frame 110. Optionally, the frame 110 can include a base 112 configured to contact a surface 116. The surface 116 can be any surface. In some implementations, the surface 116 can be a section of turf, grass, dirt, sand, or other ground covering. In some implementations, the surface 116 can be a rigid and/or fixed surface like concrete, metal, or flooring. In some implementations, the frame 110 can be moved between different types of surfaces to calibrate a force plate 140, as described herein (e.g., with reference to the method 400 shown in FIG. 4.

As used herein, the term “force plate” and “force plate 140” refers to a force plate that can be moved to different locations, for example by being picked up and placed on different surfaces. The terms “static force plate” and “embedded force plate” refer to force plates that are fixed in place and cannot be picked up and moved to different locations. Force plates and static force plates can include load cells 142 as shown in FIG. 1. Any number of load cells can be included in a force plate. Additionally, force plates can include legs 144, as shown in FIG. 1. Force plates and static force plates can also include a top plate 146 where the load is directed, and a bottom plate 148 that positions the load cells 142.

The frame 110 can include any number of supports 114 extending from the base 112.

The device 100 can further include a calibration ball 120. The calibration ball 120 can be coupled to a releaser 130. Optionally, the calibration ball is 20 lb to 30 lb. Optionally, the calibration ball 120 is 30 lb, as shown in FIG. 1. It should be understood that any mass or weight of calibration ball 120 can be used.

The releaser 130 can be coupled to one or more of the supports 114 of the frame. Optionally, the releaser can be a latch.

The releaser 130 an be configured in a “first condition” where the calibration ball 120 is coupled to the supports 114 by the releaser 130. The device 100 can also be configured in a “second condition” where the calibration ball 120 is uncoupled from the supports 114. The releaser 130 can be actuatable so that it couples and uncouples from the calibration ball 120.

In some implementations, the releaser 130 or latch is remotely actuatable. Alternatively or additionally, the releaser can be repeatably actuated from the first condition to the second condition at a repeatable speed. For example, if the releaser 130 is a latch, actuating the latch from the first condition to the second condition can be performed at a repeatable speed.

In some implementations, the releaser 130 includes a magnet. For example, the releaser 130 can include an electromagnet configured to generate a magnetic field to hold the calibration ball 120 in the first condition. Optionally, the releaser 130 is a magnet.

In some implementations, a cable 150 can be coupled to the calibration ball 120. Optionally the cable 150 can couple the calibration ball 120 to the frame 110, the supports 114 and/or to the releaser 130.

In some implementations, the distance 155 from the base 112 to the releaser 130 is adjustable. Optionally, the distance 155 from the base 112 to the releaser 130 can be a vertical distance, as shown in FIG. 1.

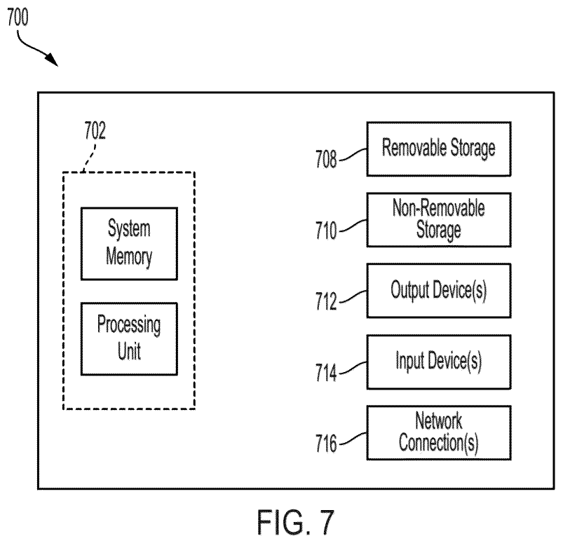

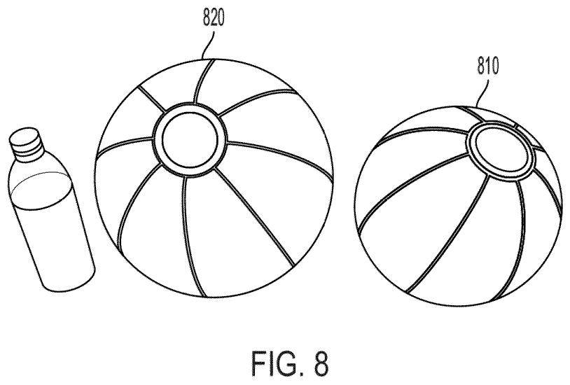

In some implementations, the device 100 can include a controller 190. The controller 190 can include any/all of the components described with reference to the computing device of FIG. 7. As shown in FIG. 7, the controller 190 can be configured to control the releaser 130. For example, the controller 190 can cause the releaser 130 to release the calibration ball 120 and/or cause the releaser 130 to attach to a calibration ball 120. Alternatively or additionally, in some implementations the controller 190 can be configured to receive data from the force plate 140.

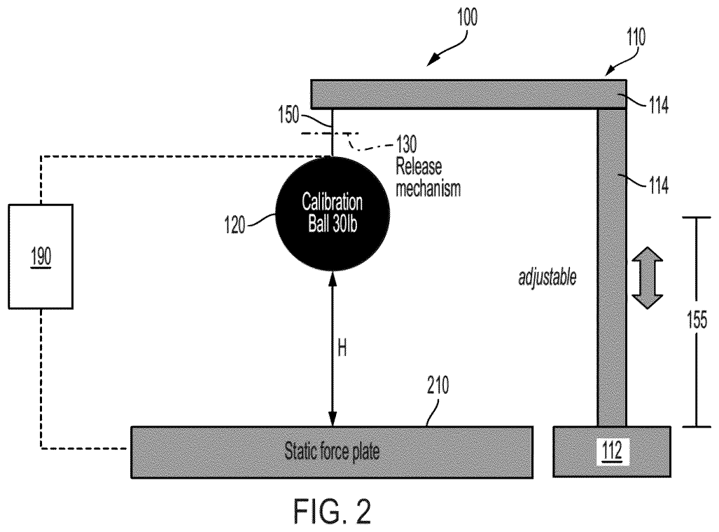

With reference to FIG. 2, the device 100 of FIG. 1 is shown with a static force plate 210. The frame 110, releaser 130, supports 114, base 112, controller 190, and calibration ball 120 are described with reference to FIG. 1. The device 100 is positioned so that the calibration ball 120 can drop onto the static force plate 210. The static force plate 210 can be a force plate that is configured so that the force of the calibration ball 120 that is dropped by the releaser 130 does not cause the force plate to move unpredictably when the calibration ball 120 impacts the force plate, and therefore the static force plate 210 can measure the entire force of the calibration ball. Thus, the static force plate 210 can be a more accurate measure of the force of the calibration ball 120 than a force plate that is not static. The device 100 can be positioned to test the force plate 140 shown in FIG. 1 and the static force plate 210 shown in FIG. 2 using the same weights/masses. By moving the device 100 and force plate 140 to different locations, it is possible to obtain force measurements for the same weight/mass dropping the same distance, onto force plates on different surfaces, and thereby determine the relationship between the surface and the force measurement using the force plate 140. The relationship between the surface 116 and the force measurement can be used to calibrate the force plate 140 for different surfaces (e.g., using the controller 190, shown in FIGS. 1 and 2). Examples of calibration are described herein, for example with reference to FIG. 4 and the examples.

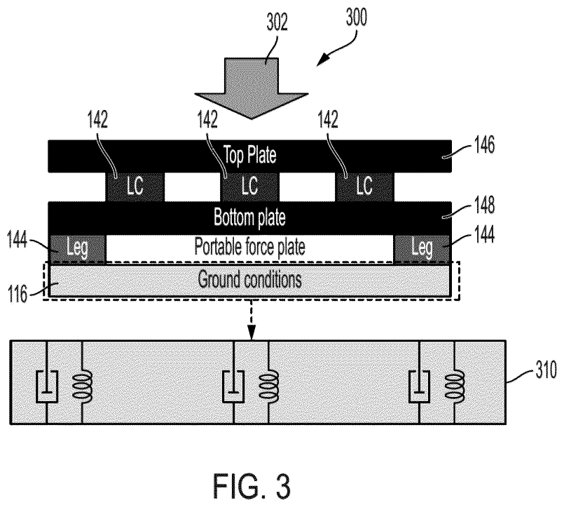

With reference to FIG. 3, a block diagram 300 is illustrated with a force 302 acting on the portable force plate 140 described with reference to FIG. 1. As shown in FIG. 3, a model 310 of the surface 116 can be created in some implementations of the present disclosure.

With reference to FIG. 4, implementations of the present disclosure include methods of calibrating a force plate. Optionally, the method 400 can be used with the device 100 of FIG. 1, and/or the device 100 of FIG. 2. Optionally, the controller 190 illustrated in FIG. 1 can be configured to implement the method steps described herein.

At step 410, the method can include obtaining any of the elements illustrated with reference to FIGS. 1 and 2. In some implementations, step 410 can include obtaining the device 100 illustrated in FIG. 1 and configuring the device 100 for calibrating a force plate 140. The device 100 can include a frame 110 including a base 112 configured to contact a surface 116 on which the force plate 140 is supported and one or more supports 114 extending from the base; a calibration ball 120; and a releaser 130 coupled to the one or more supports 114, the releaser 130 further being couplable to the calibration ball 120, where the releaser 130 is actuatable from a first condition in which the calibration ball 120 is coupled to the one or more supports and a second condition in which the calibration ball is uncoupled from the one or more supports;

In some implementations, the calibration ball 120 used in the method 400 is 20 lb to 30 lb.

At step 420, the method 400 can include disposing the base of the frame of the device over a force plate such that the force plate is directly below the calibration ball coupled to the releaser in the first condition.

At step 430, the method 400 can include actuating the releaser from the first condition to the second condition such that the calibration ball is caused to fall from the releaser onto the force plate. In some implementations, actuating the releaser can be performed at a repeatable speed. For example, actuating the releaser from the first condition to the second condition can be performable or performed at a repeatable speed. For example, in implementations where the releaser is a latch, the actuation of the latch from the first condition to the second condition can be performed at a repeatable speed.

In some implementations, the releaser is a magnet, as described with reference to the releaser 130 of FIG. 1. For example, if the magnet is an electromagnet, actuating the releaser can include energizing and/or de-energizing the electromagnet.

At step 440, the method 400 can include adjusting a calibration setting of the force plate to calibrate the force plate.

In some implementations, the method 400 can further include, after the calibration ball is caused to fall from the releaser onto the force plate, comparing the force measurement from the force plate to an expected force measurement based on the device.

In some implementations, the method 400 can further include, before disposing the base of the frame of the device, calibrating the device to establish an expected force measurement.

In some implementations, the method 400 can include remotely actuating the releaser.

In some implementations, the method 400 can include using a latch as the releaser. Optionally, the method 400 can include remotely actuating the latch.

As described with reference to FIG. 1, the device used in the method 400 can include a cable coupled to the calibration ball.

In some implementations, the distance from the base to the releaser can be adjustable in the device used for the method 400. Optionally, the method 400 can include adjusting the distance form the base to the releaser.

In some implementations, the method 400 can include before adjusting a calibration setting, adjusting the distance from the base to the releaser, and actuating the releaser from the first condition to the second condition such that the calibration ball is caused to fall from the releaser onto the force plate at the adjusted distance.

In some implementations, the method 400 can include more than one calibration ball. For example, the method 400 can include a first calibration ball and a second calibration ball. The calibration ball described with reference to steps 410, 420, 430, and 440 can be the first calibration ball. The second calibration ball can be a calibration ball with a different weight or mass from the first calibration ball. Optionally, when the method 400 includes more than one calibration ball, the method can include, before adjusting a calibration setting, actuating the releaser from the first condition to the second condition such that the second calibration ball is caused to fall from the releaser onto the force plate.

### EXAMPLE

An example implementation of the present disclosure was designed and tested. A study of the example implementation was performed. The example implementation includes a transfer function to align the different surface results to the data on the embedded force plates. The example transfer function can align different surface results to the data on one or more embedded force plates. As used herein, an “embedded” force plate is a force plate that is fixed in place relative to a surface. An example of an embedded force plate is a force plate that is embedded into a floor of a structure. Optionally, an embedded force plate can be flush to the surface of the floor (i.e., the surface of the embedded force plate is at the same level as the surface of the floor the plate is embedded in).

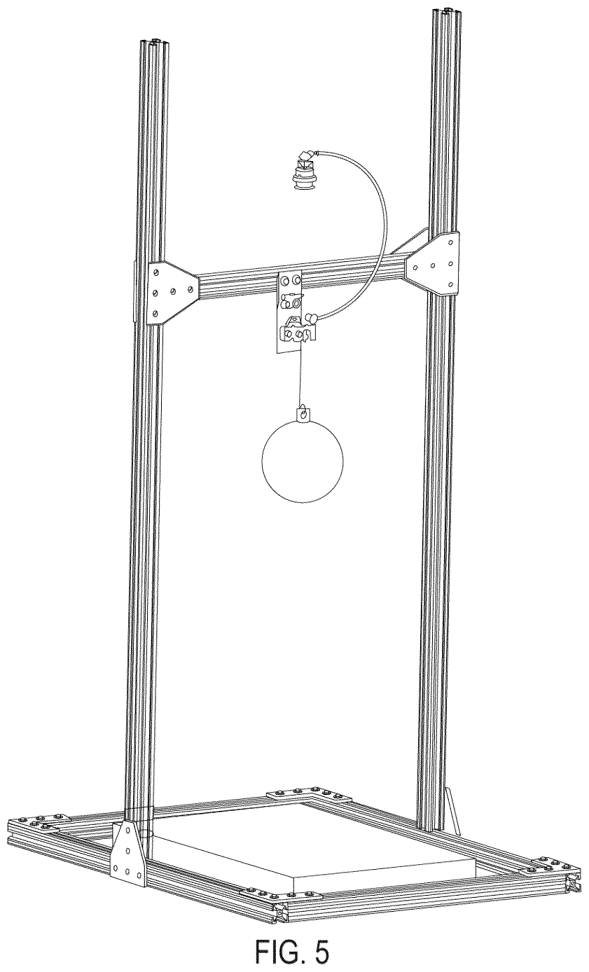

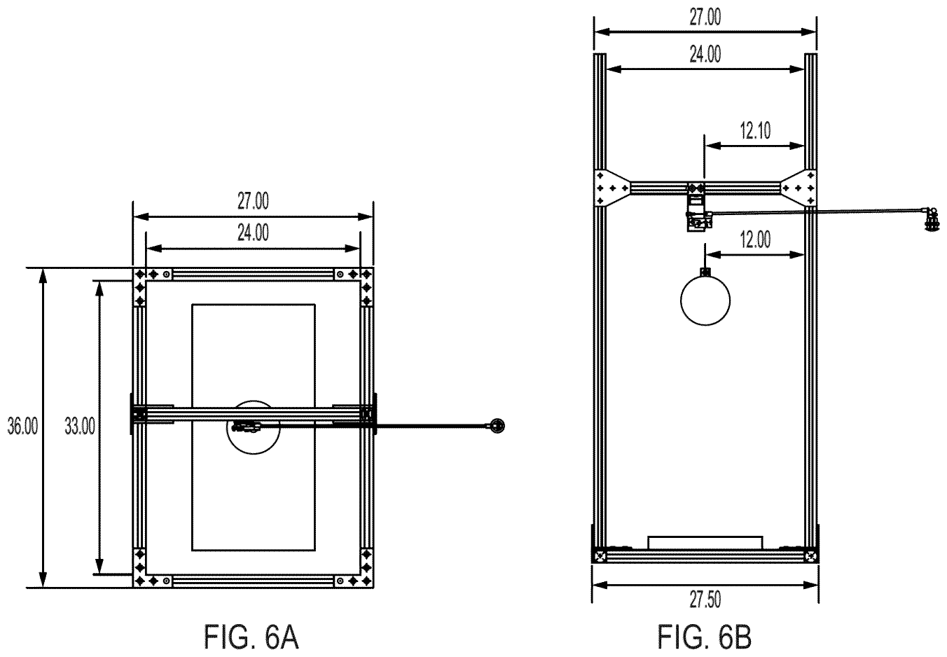

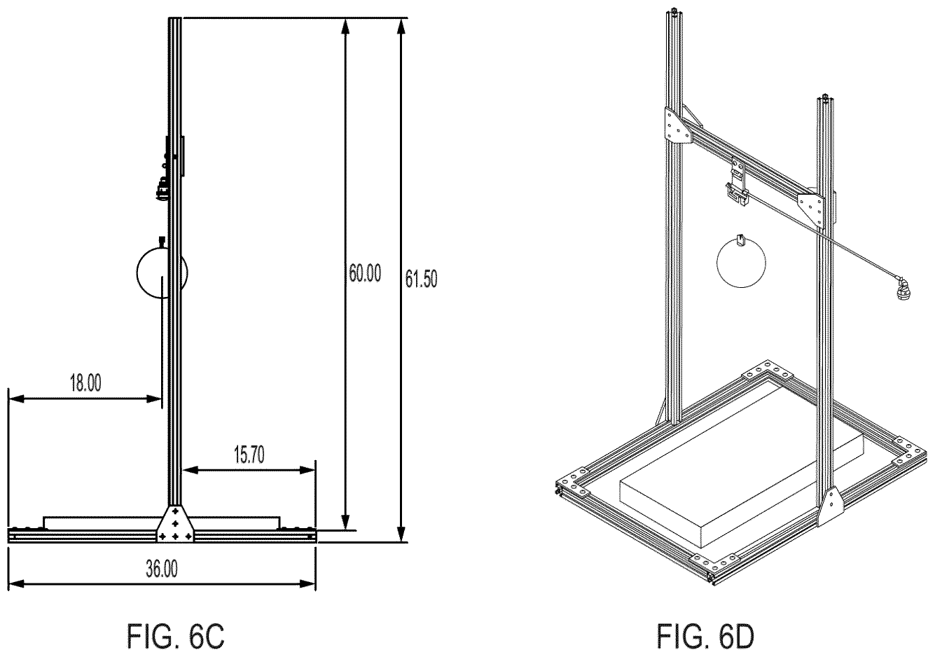

In the study, an example device was designed for the example implementation. FIG. 5 illustrates a perspective view of the example device. FIG. 6A illustrates a top view of the device illustrated in FIG. 5. FIG. 6B illustrates a first side view of the device illustrated in FIG. 5. FIG. 6C illustrates a second side view of the device illustrated in FIG. 5. FIG. 6D illustrates a perspective view of the device shown in FIGS. 5-6C. FIG. 7 illustrates an example computing device.

In the study described herein, the embedded force plate used was an embedded force plate sold under the trademark Bertec, and the force plate was a force plate sold under the trademark Vald.

The study of the example implementation included calibration to align the force curves for the force plate with the force curves with the embedded plate, when the force plate was placed on different surfaces.

The study of the example implementation included different data processing techniques as part of the calibration. Optionally, data obtained from performing force tests on the force plate on different surfaces, and/or data obtained from performing force tests on embedded forced plates, can be interpolated. Alternatively or additionally, the data can be averaged. Alternatively or additionally, data can be scaled between the different tests. For example, the force plate and the embedded force plate can optionally included different y-axis and/or x-axis magnitudes. The x-axis and/or y-axis of the embedded force plate can be scaled to match the x-axis and/or y-axis of the force plate. Alternatively or additionally, the x-axis and/or y-axis of the force plate can be scaled to match the x-axis and/or y-axis of the embedded force plate.

The study included drop tests to collect drop testing data. The force plates and embedded force plates used in the tests were zeroed before testing. Recording can be started, and an object can be dropped. Repeated drops can be performed to collect additional data. As described above, data from multiple tests can be interpolated, averaged, or otherwise processed to generate a composite test result for multiple drop tests. As a non-limiting example, 10 drop tests can be performed where an objects is dropped on each of the force plate and embedded force plate. The present disclosure also contemplates that different sampling rates can be used by the force plate and/or embedded force plate. For example, in some implementations the sample rate can be 1000 Hz.

The study further included dropping different sizes and weights on the force plate and embedded force plate. In the study, a 15 lb medicine ball and an 11 lb medicine ball were used. The 15 lb medicine ball was dropped from 86 cm above the force plate The 11 lb medicine ball was dropped from 91 cm above the force plate.

As described above, the force plate can be moved between different surfaces whereas the embedded force plate may be fixed in place. The study moved the force plate to different surfaces and performed drop tests on different surfaces. Example surfaces that were studied with the force plate include; a rolled rubber surface (on top of a basketball court); a vinyl tile (on top of concrete); an indoor track (rubber over concrete); a concrete stairwell on the top floor of a building; and a concrete stairwell on the bottom floor of a building.

FIG. 8 illustrates the 11 lb medicine ball 810 and the 15 lb medicine ball 820 that were used in the study of the example implementation described herein.

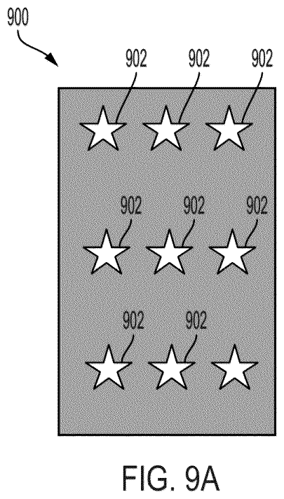

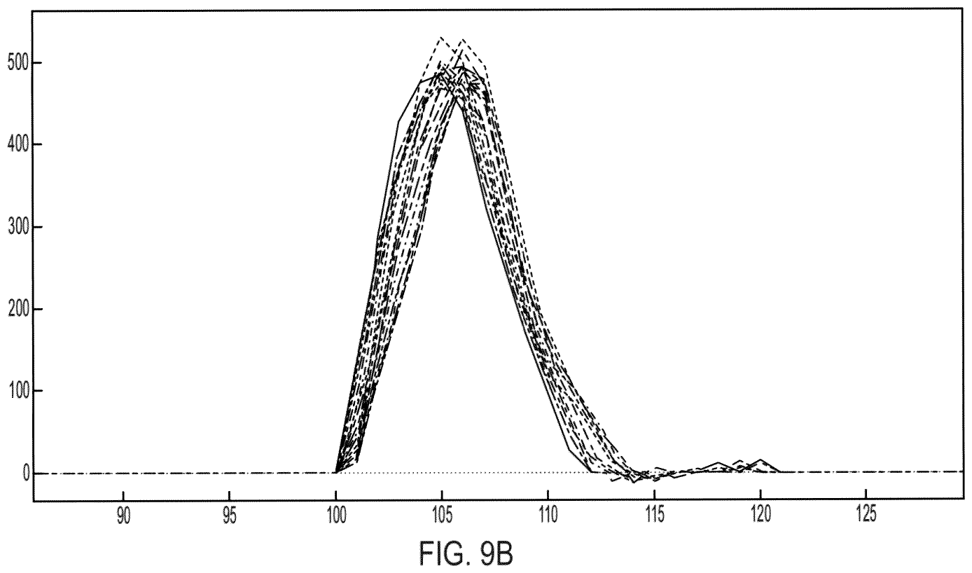

The study further included dropping a soccer ball on a force plate. The soccer ball was dropped at nine locations 902 on the force plate 900 shown in FIG. 9A. The output of drops shown in FIG. 9A is shown in FIG. 9B. FIG. 9B shows that the location of the drop on the plate does not affect the force measurement in the example implementation.

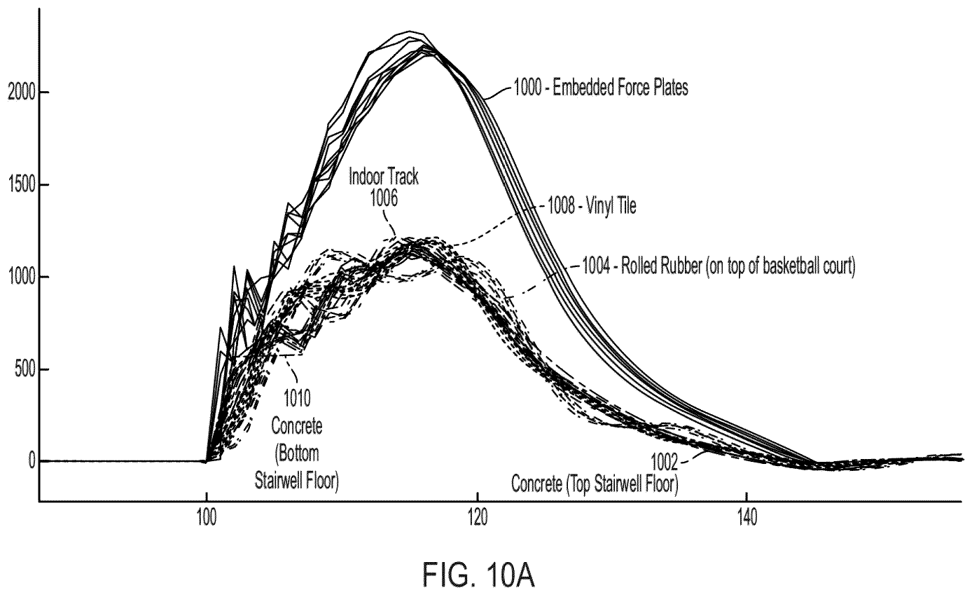

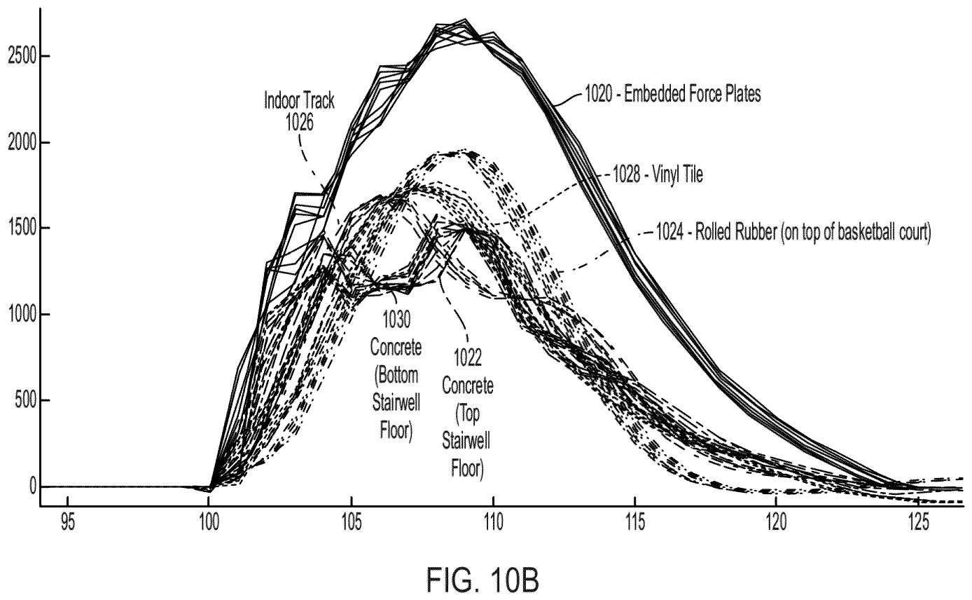

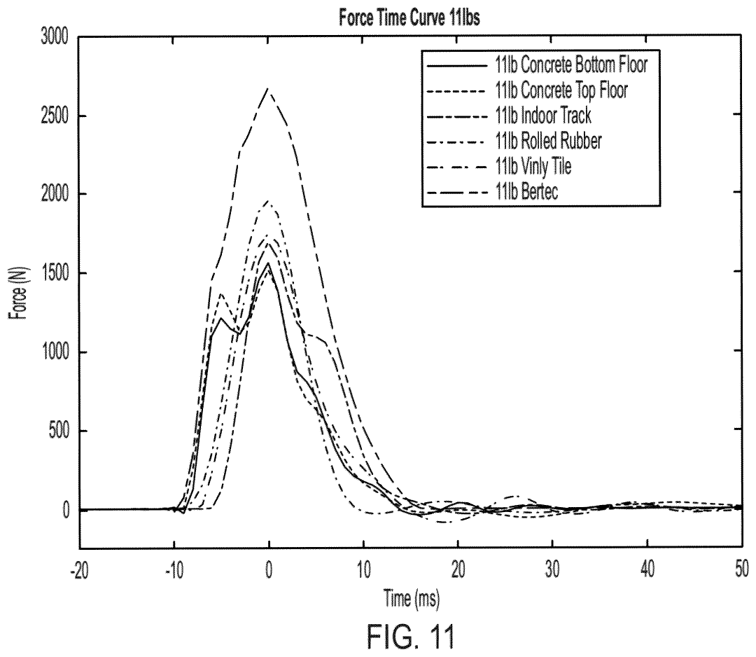

FIG. 10A includes results of force tests for different force plate locations where drop tests were conducted using a 15 lb medicine ball. FIG. 10A includes embedded force plate results 1000, concrete floor (top stairwell floor) results 1002, rolled rubber results 1004, indoor track results 1006, vinyl tile results 1008, and concrete (bottom stairwell floor) results 1010.

The embedded force plate results 1000 had a mean GRF of 2252 N and a CV of 1.81%.

The concrete floor (top stairwell floor) results 1002 had a mean GRF of 1155 N, and a CV of 1.93%.

The rolled rubber results 1004, had a mean GRF of 1126 N and a CV of 1.51%.

The indoor track results 1006, had a mean GRF of 1212 N, and a CV of 1.27%.

The vinyl tile results 1008, had a mean GRF of 1195 N and a CV of 0.79%.

The concrete (bottom stairwell floor) results 1010 had a mean GRF of 1130 N and a CV of 0.74%.

FIG. 10B illustrates results of force tests for different force plate locations where drop tests were performed using an 11 lb medicine ball. FIG. 10B includes embedded force plate results 1020, concrete floor (top stairwell floor) results 1022, rolled rubber results 1024, indoor track results 1026, vinyl tile results 1028, and concrete (bottom stairwell floor) results 1030.

The embedded force plate results 1020 had a mean GRF of 2667 N and a CV of 1.19%.

The concrete floor (top stairwell floor) results 1022 had a mean GRF of 1499 N, and a CV of 0.71%.

The rolled rubber results 1024, had a mean GRF of 1954 N and a CV of 0.51%.

The indoor track results 1026, had a mean GRF of 1684 N, and a CV of 0.59%.

The vinyl tile results 1028, had a mean GRF of 1734 N and a CV of 1.05%.

The concrete (bottom stairwell floor) results 1030 had a mean GRF of 1562 N and a CV of 1.41%.

The study included an example procedure for processing and plotting data. The example procedure included importing and filtering data. Optionally importing and/or filtering data can be performed at 50 hz. The example procedure further included interpolating the samples. Optionally, data sampled at 1000 samples per second can be interpolated to generate 20,000 samples per second. The example procedure further included cutting data to a consistent time window. The start time can be considered arbitrary. For example, the data can be cut so that 350 ms before and after the peak is used for analysis. It should be understood that the data can be cut so that different amounts of time are used for analysis, including different amounts of time before and/or after the peak, or any combinations of times. The example procedure further included averaging the trials of each different mass of weight on each different surface. The output can include a mean curve corresponding to each combination of surface and weight tested. The output pre-scaled data can be plotted.

In some implementations, the procedure can further include Y axis scaling. As described herein, the Y-axis can represent the force magnitudes, so that Y-axis scaling can include rescaling the force magnitudes. For example, the scaling factor can be defined as the maximum of the embedded force plate (e.g., the Bertec plate) and the maximum of the force plate (e.g., the Vald force plate):

$$scaling factor = \frac{max ( Bertec )}{max ( Vald )} ,$$

so that the yₐₓᵢₛ scaled force data=Vald_{force data}*scaling factor to create plotted y-scaled data. Again, the embedded force plate and force plate described with reference to the scaling factor are intended only as non-limiting examples and other force plates can be used.

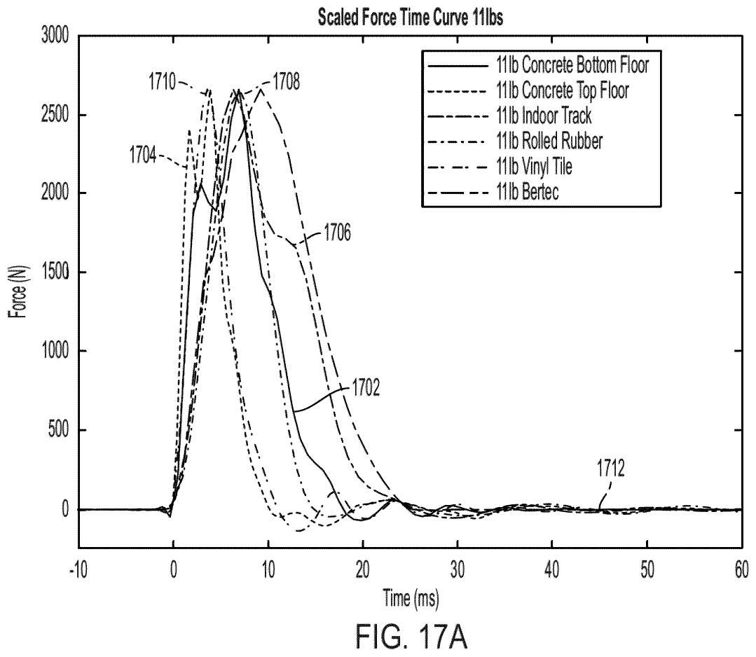

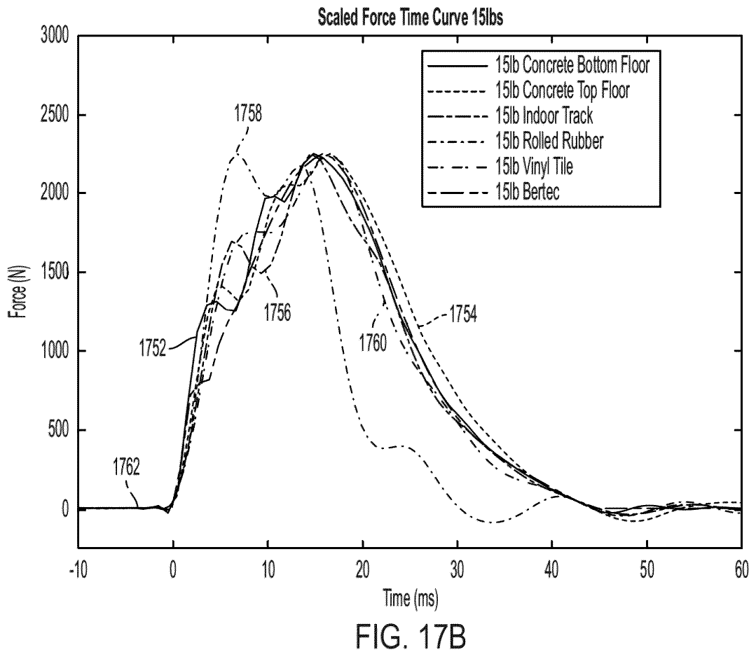

The procedure can further include aligning a point on one or more force graphs for different force tests. Optionally, the point on each of the one or more force graphs can be selected based on the first instance when a particular force is experienced. In the study, the first instance of 50 N of force on each graph was chosen as the point for each respective graph. Optionally, the X-axis of the graph can be set so that the first instance of 50 N on each graph is considered the “0” ms mark of the graph. FIG. 17A illustrates force-time curves for an 11 lb weight aligned using the first instance of 50 N on the respective curves. The curves shown in FIG. 17A include a concrete bottom floor curve 1702, a concrete top floor curve 1704, an indoor track curve 1706, a rolled rubber curve 1708, a vinyl tile curve 1710, and an embedded force plate curve 1712.

FIG. 17B illustrates force time curves for a 15 lb weight aligned using the first instance of 50 N on the respective curves. The curves shown in FIG. 17A include a concrete bottom floor curve 1752, a concrete top floor curve 1754, an indoor track curve 1756, a rolled rubber curve 1758, a vinyl tile curve 1760, and an embedded force plate curve 1762.

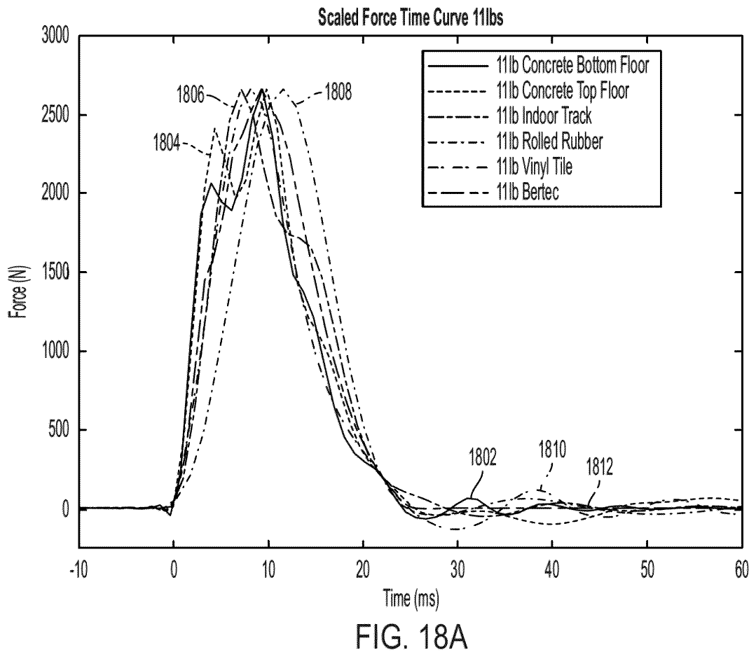

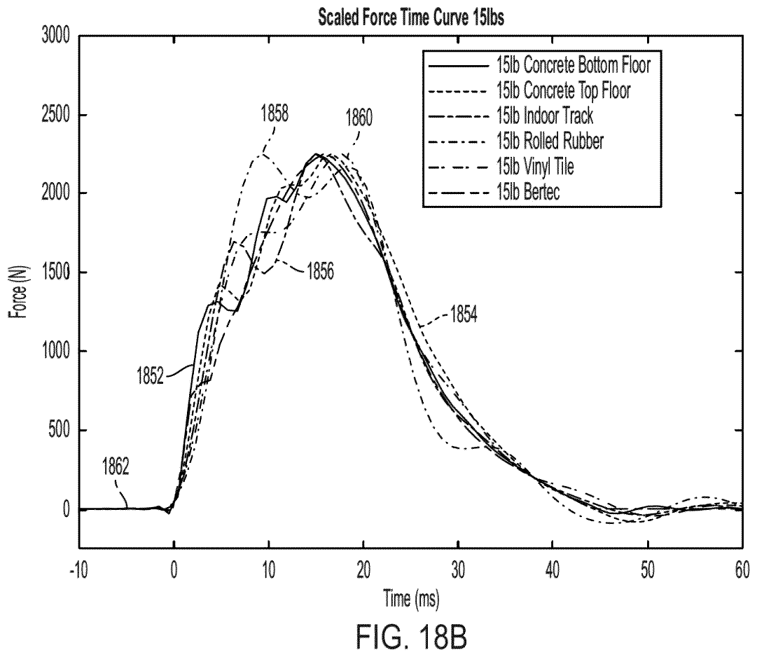

It should be understood that 50 N is only a non-limiting example, and that any amount of force can be used to set the “0” ms on the graphs. FIG. 18A illustrates force time curves for an 11 lb weight aligned using the first instance of 200 N on the respective curves.

The curves shown in FIG. 18A include a concrete bottom floor curve 1802, a concrete top floor curve 1804, an indoor track curve 1806, a rolled rubber curve 1808, a vinyl tile curve 1810, and an embedded force plate curve 1812.

FIG. 18B illustrates force time curves for a 15 lb weight aligned using the first instance of 200 N on the respective curves. The curves shown in FIG. 18B include a concrete bottom floor curve 1852, a concrete top floor curve 1854, an indoor track curve 1856, a rolled rubber curve 1858, a vinyl tile curve 1860, and an embedded force plate curve 1862.

Once the “0” for each graph has been selected, the data analyzed can be limited to data within a certain time before and after the “0” on the graph. For example, in the study described herein, data 100 ms before the point where the graph first reached 50 N and 300 ms after the graph first reached 50 N was kept and analyzed.

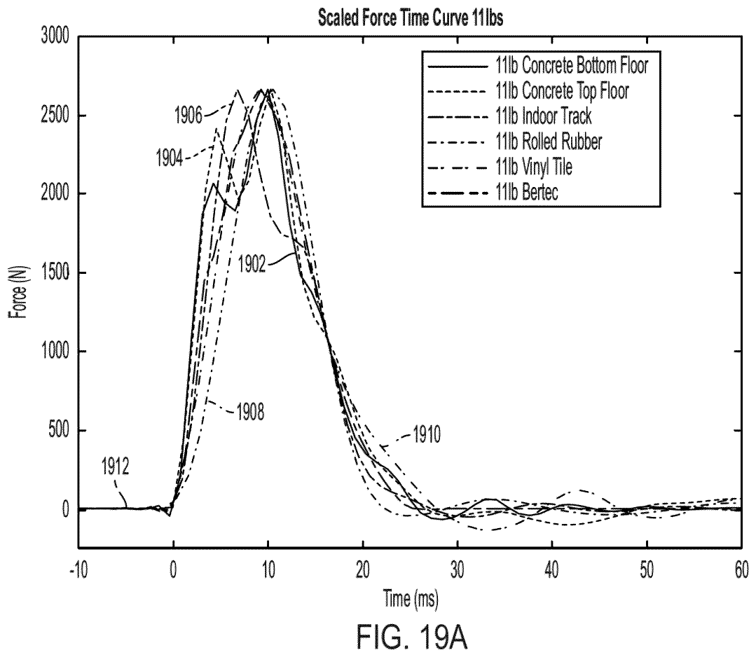

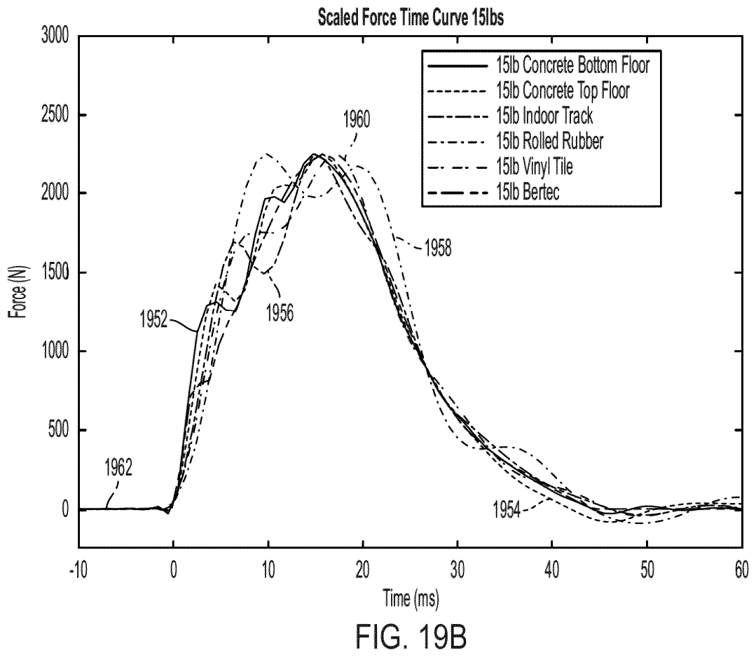

The example procedure can further include x-axis scaling. In some implementations, x-axis scaling can be performed so that the right side of a curve is set by 40% of the maximum force for the embedded force plate. It should be understood that in other implementations, different percentages of the maximum force for the embedded force plate can be used. The scaling factor can be calculated as:

$$scaling factor = \frac{time {duration}_{Bertec}}{time {duration}_{Vald}} ,$$

where again “Beretc” and “Vald” correspond to the example embedded force plate and force plate, respectively. The x-axis scaled time vector can be calculated as: xₐₓᵢₛ scaled time vector=Vald_{time vector}*scaling factor, where again “Vald” refers to the example force plate. The outputs of the force plate and embedded force plate can be output both with and without X-axis scaling and/or y-axis scaling. FIG. 19A illustrates force-time curves for an 11 lb weight, where the curves aligned using 40% of the maximum force for the embedded force plate. The curves shown in FIG. 19A include a concrete bottom floor curve 1902, a concrete top floor curve 1904, an indoor track curve 1906, a rolled rubber curve 1908, a vinyl tile curve 1910, and an embedded force plate curve 1912.

FIG. 19B illustrates force-time curves for an 15 lb weight, where the curves aligned using 40% of the maximum force for the embedded force plate. The curves shown in FIG. 19B include a concrete bottom floor curve 1952, a concrete top floor curve 1954, an indoor track curve 1956, a rolled rubber curve 1958, a vinyl tile curve 1960, and an embedded force plate curve 1962.

FIG. 11 illustrates force-time curves for an 11 lb weight without scaling.

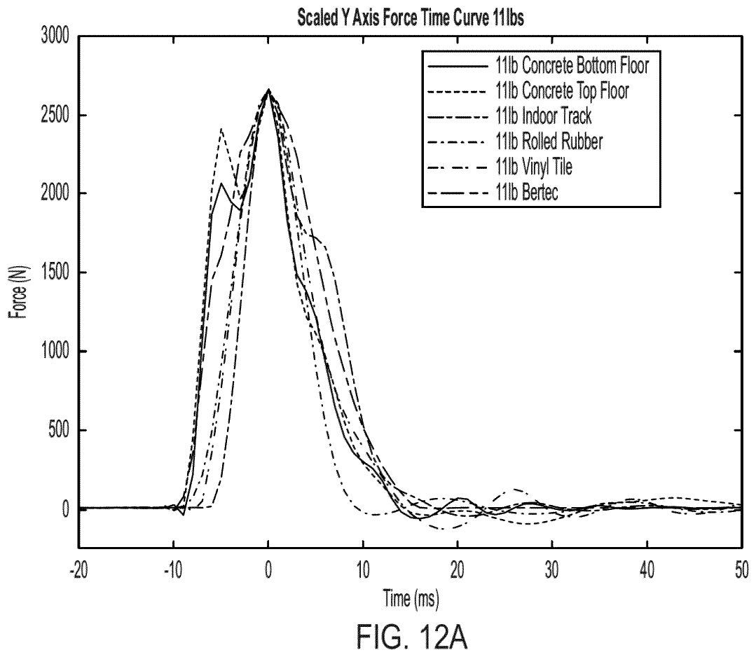

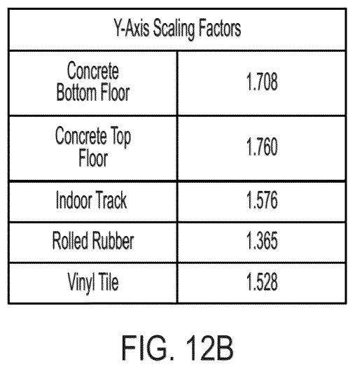

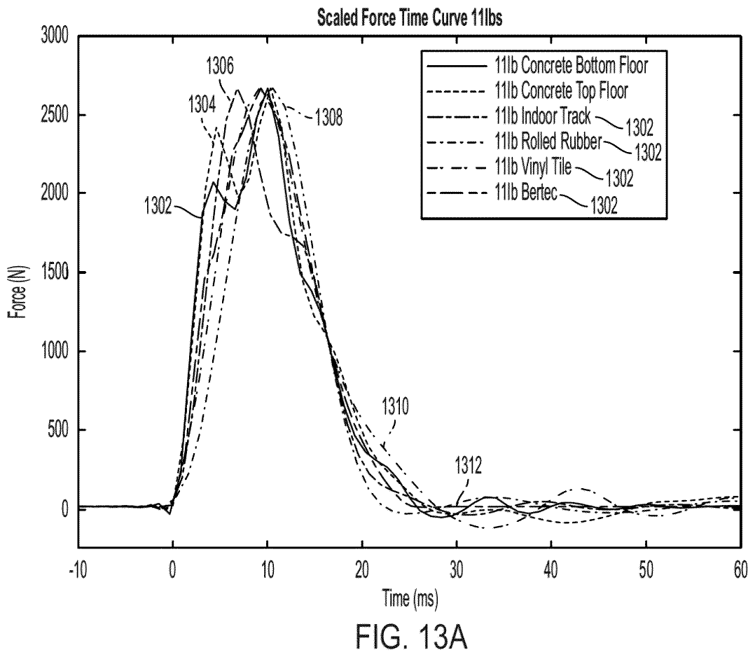

FIG. 12A illustrates force-time curves for an 11 lb weight with a scaled Y-axis. FIG. 12B illustrates Y-axis scaling factors that were applied to the curves shown in FIG. 12A.

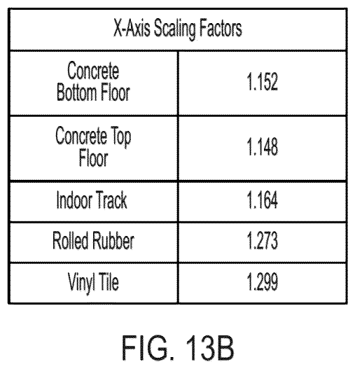

FIG. 13A illustrates force-time curves for an 11 lb weight with a scaled X-axis. The curves include a concrete bottom floor curve 1302, a concrete top floor curve 1304, an indoor track curve 1306, a rolled rubber curve 1308, a vinyl tile curve 1310, and an embedded force plate curve 1312.

FIG. 13B illustrates the X-axis scaling factors that were applied to the curves shown in FIG. 13A. The Y-axis scaling factors that were applied to the curves shown in FIG. 13A are shown in FIG. 12B.

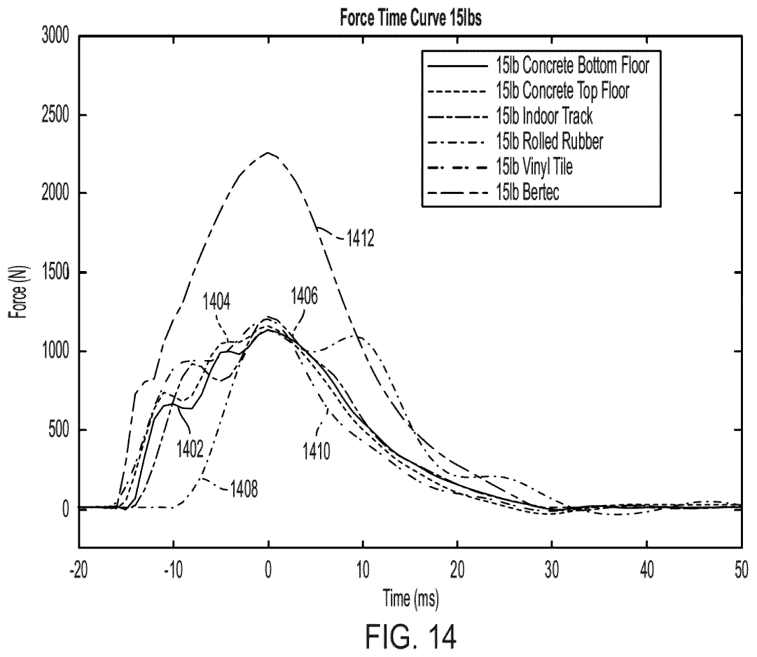

FIG. 14 illustrates force-time curves for a 15 lb weight without scaling. The curves include a concrete bottom floor curve 1402, a concrete top floor curve 1404, an indoor track curve 1406, a rolled rubber curve 1408, a vinyl tile curve 1410, and an embedded force plate curve 1412.

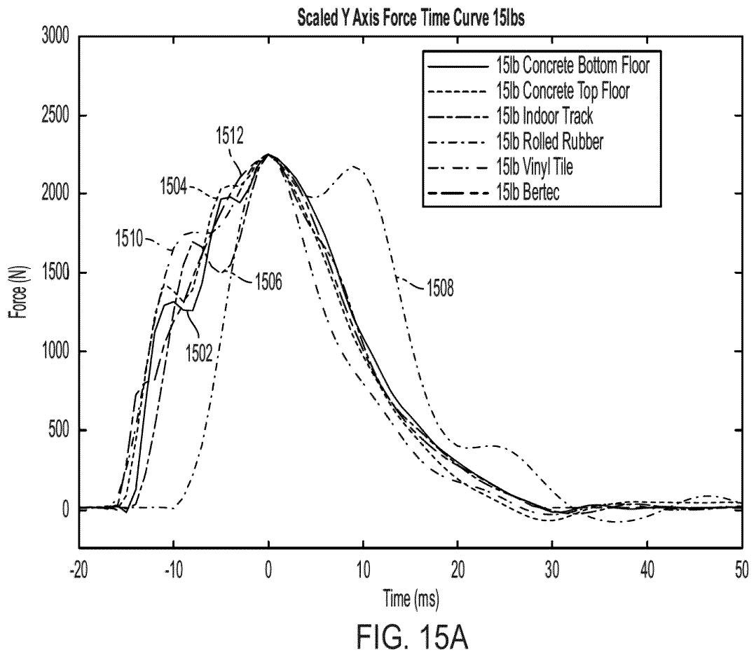

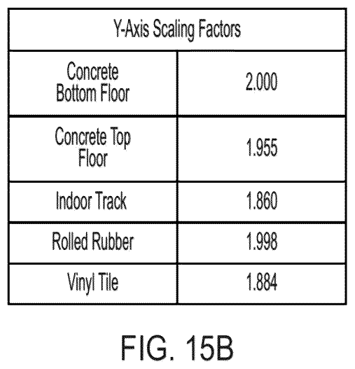

FIG. 15A illustrates force time-curves for the 15 lb weight with a scaled Y-axis. FIG. 15B illustrates the Y-axis scaling factors that were applied to the curves shown in FIG. 15A. The curves include a concrete bottom floor curve 1502, a concrete top floor curve 1504, an indoor track curve 1506, a rolled rubber curve 1508, a vinyl tile curve 1510, and an embedded force plate curve 1512.

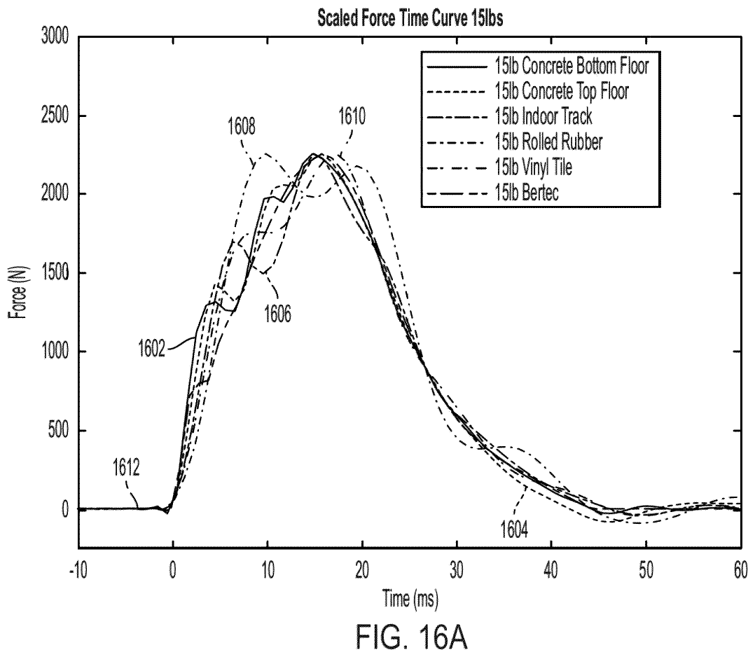

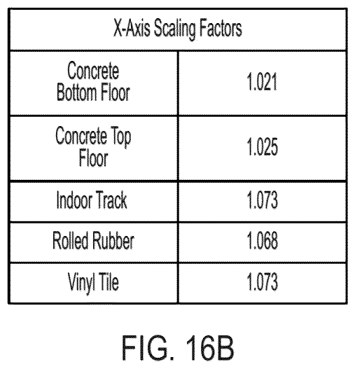

FIG. 16A illustrates force-time curves for the 15 lb weight with a scaled X-axis and a scaled Y-axis. FIG. 16B illustrates the X-axis scaling factors that were applied to the curves shown in FIG. 16A. The Y-axis scaling factors that were applied to the curves shown in FIG. 16A are shown in FIG. 15B. The curves include a concrete bottom floor curve 1602, a concrete top floor curve 1604, an indoor track curve 1606, a rolled rubber curve 1608, a vinyl tile curve 1610, and an embedded force plate curve 1612.

In some implementations, the study can include fixtures for pre-loading the force plate with the fixture.

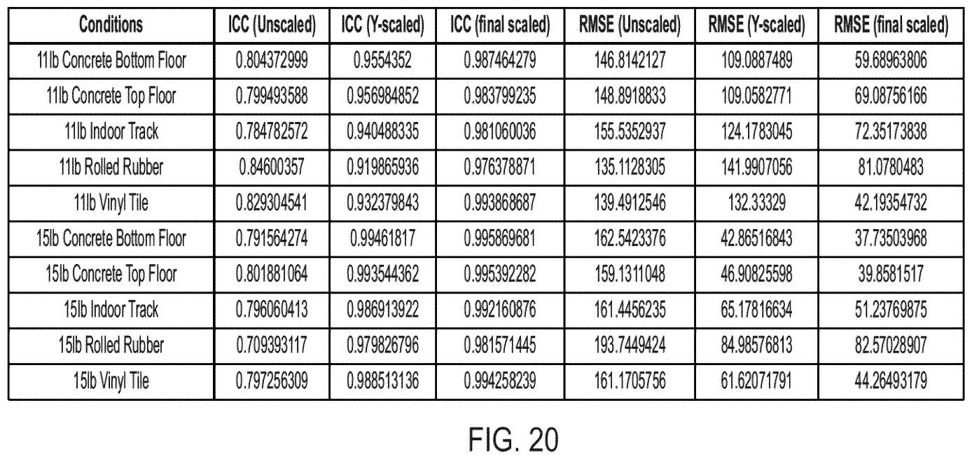

The example implementation of the present disclosure was further studied using an analysis of Intraclass Correlation Coefficients (ICC) and root-mean-square error (RMSE) values. The ICC and RMSE values can quantify (1) the agreement between the ground reaction force data measured by the embedded force plate and the ground reaction force data measured by the portable force plate, and/or (2) the average difference between the ground reaction force measurement of the two systems at any given time point during a data collection. FIG. 20 illustrates example ICC and RMSE values for unscaled and scaled test results. ICC values of 1.00 can represent a perfect agreement between two systems (e.g., the results from a force plate on different surfaces and an embedded force plate). Alternatively or additionally, an ICC value of >0.9 can be considered a strong agreement between the two values. RMSE values shown in FIG. 20 are expressed in terms of Newtons of force. Lower RMSE values can represent a closer agreement (or “fit”) between two results (e.g., the results from a force plate on different surfaces and an embedded force plate). RMSE values given herein are in terms of Newtons (N).

It should be appreciated that the logical operations described herein with respect to the various figures may be implemented (1) as a sequence of computer implemented acts or program modules (i.e., software) running on a computing device (e.g., the computing device described in FIG. 7), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 7, an example computing device 700 upon which the methods described herein may be implemented is illustrated. It should be understood that the example computing device 700 is only one example of a suitable computing environment upon which the methods described herein may be implemented. Optionally, the computing device 700 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 700 typically includes at least one processing unit 706 and system memory 704. Depending on the exact configuration and type of computing device, system memory 704 may be volatile (such as random access memory (RAM)), non-volatile (such as read-only memory (ROM), flash memory, etc.), or some combination of the two. This most basic configuration is illustrated in FIG. 7 by dashed line 702. The processing unit 706 may be a standard programmable processor that performs arithmetic and logic operations necessary for operation of the computing device 700. The computing device 700 may also include a bus or other communication mechanism for communicating information among various components of the computing device 700.

Computing device 700 may have additional features/functionality. For example, computing device 700 may include additional storage such as removable storage 708 and non-removable storage 710 including, but not limited to, magnetic or optical disks or tapes. Computing device 700 may also contain network connection(s) 716 that allow the device to communicate with other devices. Computing device 700 may also have input device(s) 714 such as a keyboard, mouse, touch screen, etc. Output device(s) 712 such as a display, speakers, printer, etc. may also be included. The additional devices may be connected to the bus in order to facilitate communication of data among the components of the computing device 700. All these devices are well known in the art and need not be discussed at length here.

The processing unit 706 may be configured to execute program code encoded in tangible, computer-readable media. Tangible, computer-readable media refers to any media that is capable of providing data that causes the computing device 700 (i.e., a machine) to operate in a particular fashion. Various computer-readable media may be utilized to provide instructions to the processing unit 706 for execution. Example tangible, computer-readable media may include, but is not limited to, volatile media, non-volatile media, removable media and non-removable media implemented in any method or technology for storage of information such as computer readable instructions, data structures, program modules or other data. System memory 704, removable storage 708, and non-removable storage 710 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 706 may execute program code stored in the system memory 704. For example, the bus may carry data to the system memory 704, from which the processing unit 706 receives and executes instructions. The data received by the system memory 704 may optionally be stored on the removable storage 708 or the non-removable storage 710 before or after execution by the processing unit 706.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

A number of example implementations are provided herein. However, it is understood that various modifications can be made without departing from the spirit and scope of the disclosure herein. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. Although the terms “comprising” and “including” have been used herein to describe various implementations, the terms “consisting essentially of” and “consisting of” can be used in place of “comprising” and “including” to provide for more specific implementations and are also disclosed.

Disclosed are materials, systems, devices, methods, compositions, and components that can be used for, can be used in conjunction with, can be used in preparation for, or are products of the disclosed methods, systems, and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein. For example, if a device is disclosed and discussed each and every combination and permutation of the device are disclosed herein, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed systems or devices. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

What is claimed is:

1. A device for calibrating a force plate, the device comprising:
a frame including a base configured to contact a surface on which the force plate is supported and one or more supports extending from the base;
a calibration ball; and
a releaser coupled to the one or more supports, the releaser further being couplable to the calibration ball, wherein the releaser is actuatable from a first condition in which the calibration ball is coupled to the one or more supports and a second condition in which the calibration ball is uncoupled from the one or more supports.

2. The device of claim 1, wherein the releaser is a latch.

3. The device of claim 2, wherein the latch is remotely actuatable.

4. The device of claim 3, wherein actuation of the latch from the first condition to the second condition is performable at a repeatable speed.

5. The device of claim 1, wherein the releaser is a magnet.

6. The device of claim 1, further comprising a cable coupled to the calibration ball.

7. The device of claim 1, wherein a distance from the base to the releaser is adjustable.

8. The device of claim 1, wherein the calibration ball is 20 lb to 30 lb.

9. A method of calibrating a force plate, the method comprising:
obtaining a device for calibrating a force plate, the device comprising:
a frame including a base configured to contact a surface on which the force plate is supported and one or more supports extending from the base;
a calibration ball; and
a releaser coupled to the one or more supports, the releaser further being couplable to the calibration ball, wherein the releaser is actuatable from a first condition in which the calibration ball is coupled to the one or more supports and a second condition in which the calibration ball is uncoupled from the one or more supports;
disposing the base of the frame of the device over a force plate such that the force plate is directly below the calibration ball coupled to the releaser in the first condition;
actuating the releaser from the first condition to the second condition such that the calibration ball is caused to fall from the releaser onto the force plate; and
adjusting a calibration setting of the force plate to calibrate the force plate.

10. The method of claim 9, further comprising, after the calibration ball is caused to fall from the releaser onto the force plate, comparing a force measurement from the force plate to an expected force measurement based on the device.

11. The method of claim 9, further comprising, before disposing the base of the frame of the device, calibrating the device to establish an expected force measurement.

12. The method of claim 9, wherein the releaser is a latch.

13. The method of claim 12, wherein the latch is remotely actuatable.

14. The method of claim 13, wherein actuation of the latch from the first condition to the second condition is performable at a repeatable speed.

15. The method of claim 9, wherein the releaser is a magnet.

16. The method of claim 9, wherein the device further comprises a cable coupled to the calibration ball.

17. The method of claim 9, wherein a distance from the base to the releaser is adjustable.

18. The method of claim 17, further comprising, before adjusting a calibration setting, adjusting the distance from the base to the releaser, and actuating the releaser from the first condition to the second condition such that the calibration ball is caused to fall from the releaser onto the force plate at the adjusted distance.

19. The method of claim 9, wherein the calibration ball is 20 lb to 30 lb.

20. The method of claim 19, wherein the calibration ball is a first calibration ball and the device further comprises a second calibration ball having a different weight than the first calibration ball, wherein the method further comprises, before adjusting a calibration setting, actuating the releaser from the first condition to the second condition such that the second calibration ball is caused to fall from the releaser onto the force plate.
